(19) United States
(12) Patent Application Publication
(10) Pub. No.: US 2015/0173763 A1
(43) Pub. Date: Jun. 25, 2015

(54) Sandwich-type Gastrointestinal Anastomosis Stapler

(71) Applicant: Zhongchen Liu, Xiamen (CN)

(72) Inventor: Zhongchen Liu, Xiamen (CN)

(21) Appl. No.: 13/702,162

(22) PCT Filed: Sep. 10, 2012
(86) PCT No.: PCT/CN12/81213

§ 371 Date: Dec. 5, 2012

(30) Foreign Application Priority Data
Aug. 28, 2012 (CN) 201210313148.1

(51) Int. Cl. A61B 17/115; A61B 17/32; A61B 17/068

(52) U.S. Cl. CPC A61B 17/115; A61B 17/068; A61B 17/32

(57) ABSTRACT

In one aspect of the present invention, a sandwich-type surgical structure for joining tissue includes a movable handle portion; a body portion extending distally from the handle portion; a nail assembly operatively connected to a distal end of the body portion and a first membrane-like structure; the nail assembly includes a first member; a second member; a first group of nails; a second groups of nails, and the length of the nail of the first group is in the range of 5-95% length of the second group of nail, so that the force applying to the tissue will vary depending on the length of the nails, and the first membrane-like structure immediately contacts with the tissue contacting surface of the first member of the nail assembly.

### CROSS-REFERENCE TO RELATED APPLICATIONS

This application is a continuation of U.S. patent application Ser. No. 13/702,162, filed Dec. 5, 2012, which is a national stage filing under 35 U.S.C. 371 of PCT/CN12/81213, filed Sep. 10, 2012, which claims priority to Chinese Patent Application No. 201220431263.4 and 201210313148.1, filed Aug. 28, 2012, owned by the inventor of the present invention, all of which are incorporated herein by reference in their entirety.

### FIELD OF THE INVENTION

The present invention relates to generally to a surgical structure, and more particularly, to a sandwich-type gastrointestinal anastomosis stapler for joining tissue.

### BACKGROUND OF THE INVENTION

In the gastrointestinal surgery, often requires the reconstruction of the digestive tract suture and after suturing the excess tissue in organ must be removed to reconstruct the digestive tract. In order to ensure the successful completion of the surgery, usually use one-time use of gastrointestinal stapler, currently used surgical instrument, “a one-time use of gastrointestinal stapler”, it's design principles commonly used stapler theory, there are tubular and linear two categories, mainly composed by nail anvil (also known as needle seat), nail warehouse (also called nail box), the body, mobile handle, adjusting knob, shim and suture nail (also called anastomosis nail), in addition comes with a cutter stapler to be a connecting rod. In the gastrointestinal surgery, through the procession of two rows of suture nails along the body in stagger order are docking the organ in digestive tract and the procession of the central circular cutter cutting the sutured excess tissues to complete the reconstruction of the digestive tract, When the tissues in digestive tract has accurately come in the connection part of the nail anvil and the body's front part and effectively hold by the joints, as well as the safe button rotate smoothly as required, although control the handle, proceed to firing and implement docking the organ in the digestive tract, but there are advantages such as less anastomotic tear, anastomotic leakage and anastomotic bleeding, these side effect will cause problem to a patient and cast negative images for health care professionals.

China Patent CN1695563 discloses a gastrointestinal tubular stapler, as far as to use buckle structure material for connection of plastic parts with plastic parts and plastic parts with metal parts to adapt to the characteristics of plastic materials, by changing the anvil section shape to minimize the resistance from anvil cutter and firing mechanism of gastrointestinal tube stapler. By using of tooth structure, the safety mechanism except has a role of safety, also has a role of voice prompts to sign complete of suture and a role of anti-open the mobile handle during clamping and firing, indicate more detailed nail anvil's position through instruction rulers on the window.

China Patent CN1973782 discloses a gastrointestinal tubular stapler, using the stop block on inside surface of the cutter anvil make nail anvil can not rotate around the bar. When suturing, circular cutter cut the section between nail anvil's inner side and outer side, the drive some parts of inner side of cutter anvil detach from the nail anvil, with incline spring action the nail anvil rotate incline around the nail anvil bar, which can easily remove the digestive tubular stapler.

China patent CN101856250A disclosed a one-time auto-safety circular stapler, including nail anvil components, nail box components, aluminum bend pipe, bend putter components, mobile handle components, adjustment nut components, automatic safety device and a fixed handle components; bend putter components including bend putter, Shaft, connecting piece, nail rod, adjusting block, adjusting nail and compression chip; set bent putter components in bent aluminum pipe, nail box components including nail set, nail box, push piece and circular cutter; nail anvil components including nail anvil, spring tube, spring and nail anvil lid; automatic safety device including safety positioning plate, safety positioning reset plate, safety positioning reset spring, safety positioning reset spring pin, indicating block, indicating block transmission plate, indicating block transmission plate's reset spring and indicating block transmission plate positioning pin.

Therefore, a heretofore unaddressed need exists in the art to address the aforementioned deficiencies and inadequacies.

### SUMMARY OF THE INVENTION

The present invention, in one aspect, relates to a sandwich-type gastrointestinal anastomosis stapler for joining tissue.

In one embodiment, the sandwich-type surgical structure for joining tissue including a movable handle portion; a body portion extending distally from the handle portion; a nail assembly operatively connected to a distal end of the body portion, the nail assembly comprising: a first member; a second member; a first group of nails; a second groups of nails, wherein the length of the nail of the first group is in the range of 5-95% length of the second group of nail, so that the force applying to the tissue will vary depending on the length of the nails; and a first membrane-like structure wherein the first membrane-like structure immediately contacts with the tissue contacting surface of the first member of the nail assembly. In another embodiment, the length of the nail of the first group is in the range of 35-95% length of the second group of nail, so that the force applying to the tissue will vary depending on the length of the nails. In one embodiment, the first membrane-like structure, the associated intestine walls, and the second membrane-like structure forming a sandwich structure comprising a membrane-like structure, the associated intestine walls, and the membrane-like structure. In another embodiment, the described membrane-like structure is an annular or a strip shaped membrane. In one embodiment, the annular shaped membrane has a width of 1.5˜3.0 mm, and a thickness of 0.1˜1.0 mm. In another embodiment, the strip shaped membrane has a width of 1.5˜5 mm, and a thickness of 0.1˜1.0 mm. In one embodiment, the membrane-like structure has both flexibility and rigidity. In another embodiment, the membrane-like structure with flexibility and rigidity is a degradable polymer. In one embodiment, the membrane-like structure with flexibility and rigidity is a non-degradable polymer. In another embodiment, the membrane-like structure is one of the silicone, polymer milk alcohols, poly lactic acid, polyoxyethylene, polyester material.

In one embodiment, the sandwich-type surgical structure further comprising: a second membrane-like structure wherein the second membrane-like structure immediately contacts with the tissue contacting surface of the second member of the nail assembly.

In another embodiment, the sandwich-type surgical structure further comprising: cutter for cutting excess tissues from an associated digestive tract to open up a central conduit to complete the reconstruction of the associated digestive tract; wherein the first and second group of nails are linear arrays, wherein the linear arrays parallel to each other; wherein the first and second linear arrays are interposing, wherein the second member is operatively associated with the first member, the first and second members being movably connected to one another to bring one into longitudinal juxtaposition relative to the other.

In one embodiment, a sandwich-type surgical structure for joining tissue, comprising: The sandwich-type surgical structure for joining tissue including a movable handle portion; a body portion extending distally from the handle portion; a nail assembly operatively connected to a distal end of the body portion, the nail assembly comprising: a first member; a second member; a first group of nails; a second groups of nails, wherein the length of the nail of the first group is in the range of 5-95% length of the second group of nail, so that the force applying to the tissue will vary depending on the length of the nails. In one embodiment, the length of the nail of the first group is in the range of 35-95% length of the second group of nail, so that the force applying to the tissue will vary depending on the length of the nails. In another embodiment, the length of the nail of the first group is in the range of 65-85% length of the second group of nail, so that the force applying to the tissue will vary depending on the length of the nails. In one embodiment, the first member is a nail cartridge assembly and the second member is a nail anvil assembly operatively associated with the nail cartridge assembly.

In another embodiment, the sandwich-type surgical structure further comprising: cutter for cutting excess tissues from an associated digestive tract to open up a central conduit to complete the reconstruction of the associated digestive tract; wherein the first and second group of nails are linear arrays, wherein the linear arrays parallel to each other; wherein the first and second linear arrays are interposing, wherein the second member is operatively associated with the first member, the first and second members being movably connected to one another to bring one into longitudinal juxtaposition relative to the other.

In one embodiment, a sandwich-type surgical structure for joining tissue includes a movable handle portion; a body portion extending distally from the handle portion; a nail assembly operatively connected to a distal end of the body portion, the nail assembly comprising: a first member; a second member; a first group of nails; a second groups of nails; and a first membrane-like structure wherein the first membrane-like structure immediately contacts with the tissue contacting surface of the first member of the nail assembly, wherein the first membrane-like structure has at least two thicknesses. In another embodiment, the length of the nail of the first group is in the range of 5-95% length of the second group of nail, so that the force applying to the tissue will vary depending on the length of the nail and the width of the membrane-like structure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment, and wherein:

FIG. 1 shows schematically a cross-sectional view, respectively, of a conventional disposable tubular gastrointestinal stapler.

FIG. 2 shows schematically a cross-sectional view, respectively, of a conventional disposable gastrointestinal stapler structure schema.

FIG. 3 shows schematically a cross-sectional view, respectively, of a conventional disposable gastrointestinal outer-abdominal linear cutting stapler.

FIG. 4 shows schematically a cross-sectional view, respectively, of a conventional disposable gastrointestinal inner-abdominal linear cutting stapler.

FIG. 5 show schematically cross-sectional views of structure formed after stapling by the conventional gastrointestinal anastomosis stapler.

FIG. 6A-6B show schematically cross-sectional views of embodiment of the present invention (for linear sandwich gastrointestinal anastomosis stapler) the anvil; the nail assembly and membrane-like structures according to one embodiment of the present invention;

FIG. 7 shows schematically a cross-sectional view of the sandwich-type structure formed after stapling by a sandwich-type gastrointestinal anastomosis stapler according to one embodiment of the present invention.

FIG. 8 shows schematically a cross-sectional view of the sandwich-type structure formed after stapling by a sandwich-type gastrointestinal anastomosis stapler with two groups of nails according to one embodiment of the present invention.

FIG. 9 shows schematically a perspective view of the sandwich-type structure formed after stapling by a sandwich-type gastrointestinal anastomosis stapler with two groups of nails according to one embodiment of the present invention.

FIG. 10 shows schematically a cross-sectional view of the sandwich-type structure formed after stapling by a sandwich-type gastrointestinal anastomosis stapler with two groups of nails according to one embodiment of the present invention;

FIG. 11A show schematically perspective views of (for annular stapler-type sandwich gastrointestinal anastomosis cutting stapler) the nail anvil; nail assembly and membrane-like's structure according to one embodiments of the present invention;

FIG. 11B show schematically cross-sectional views of (for annular stapler-type sandwich gastrointestinal anastomosis cutting stapler) the nail anvil; nail assembly and membrane-like's structure according to one embodiments of the present invention;

FIGS. 12A and 12B shows schematically a perspective view of the sandwich-type structure formed after stapling by a sandwich-type gastrointestinal anastomosis stapler with two groups of nails according to one embodiment of the present invention;

FIG. 12C shows schematically a cross-sectional view of the sandwich-type structure formed after stapling by a sandwich-type gastrointestinal anastomosis stapler with two different thicknesses of the membrance-like structure according to one embodiment of the present invention.

### DETAILED DESCRIPTION OF THE INVENTION

The present invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

As shown in the drawings and as used in this description, and as is traditional when referring to relative positioning on an object, the term “proximal” refers to that portion of the device, or component thereof, closer to the user and the term “distal” refers to that portion of the device, or component thereof, farther from the user.

This description may use the phrases “in an embodiment,” “in embodiments,” “in some embodiments,” or “in other embodiments,” which may each refer to one or more of the same or different embodiments in accordance with the present disclosure. For the purposes of this description, a phrase in the form “AB” means A or B. For the purposes of the description, a phrase in the form “A and/or B” means “(A), (B), or (A and B)”. For the purposes of this description, a phrase in the form “at least one of A, B, or C” means “(A), (B), (C), (A and B), (A and C), (B and C), or (A, B and C)”.

As used herein, the term “membrane-like structure” generally refers to a material used surgically to join tissues. The membrane-like structure may be constructed from a variety of materials including, but not limited to, surgical gut; silk; polyolefins such as polyethylene and polypropylene; polyamides; polyesters (including aliphatic polyesters); or any combinations of the above materials adapted for use to join tissues. The membrane-like structure may be either absorbable or non-absorbable. The membrane-like structure may be conditioned, treated and or processed to make it suitable for use at internal body sites, according to means within the purview of those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom”, “upper” or “top,” and “front” or “back” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The term “nail”, as used herein, refers to suture structure such as nail and staple.

As used herein, the term “plurality” means a number greater than one.

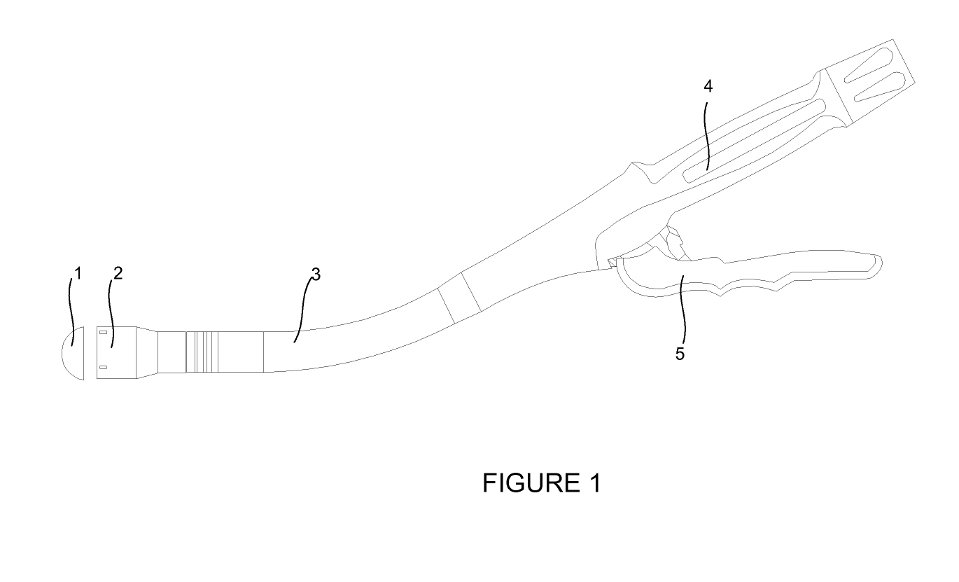

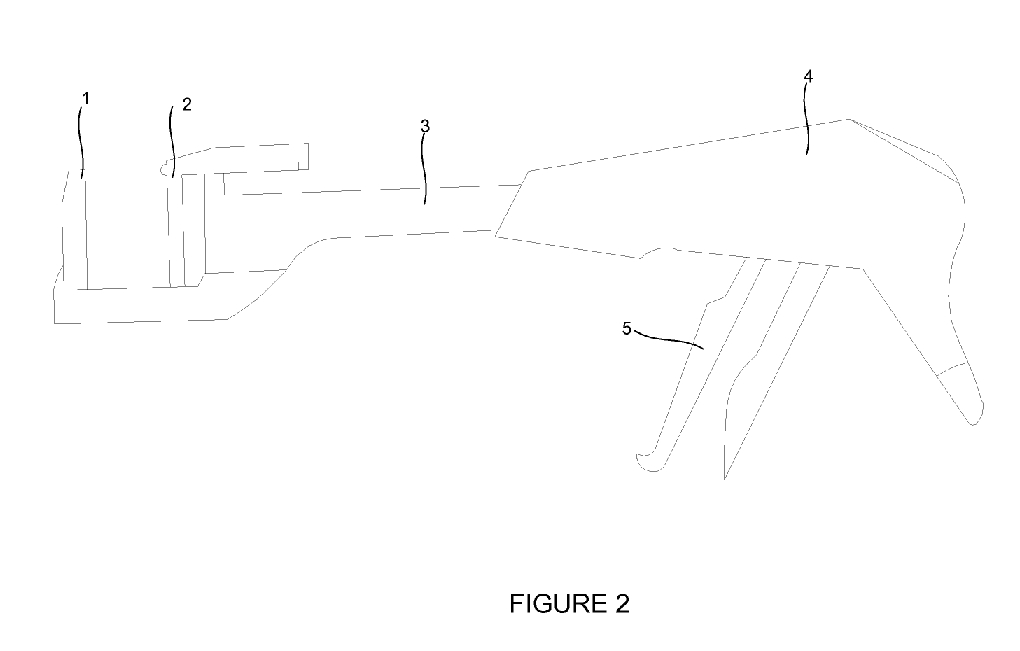

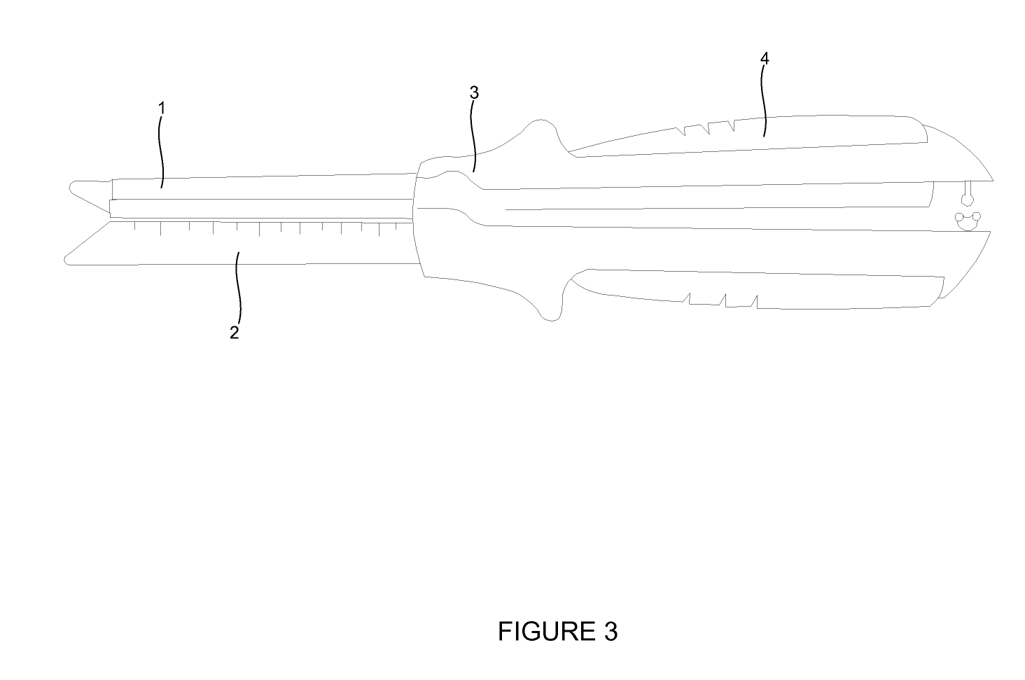

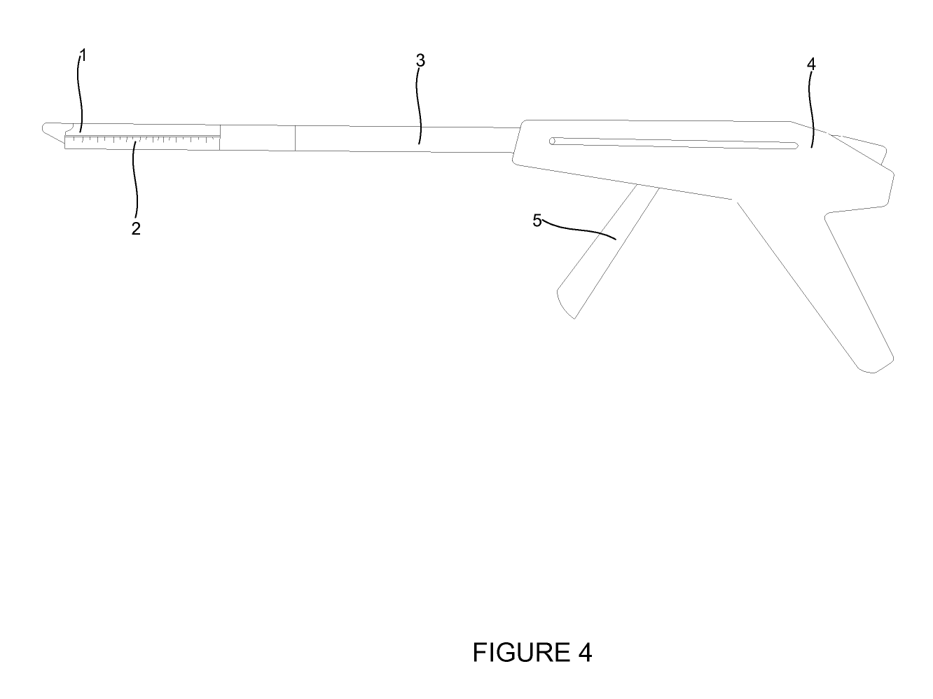

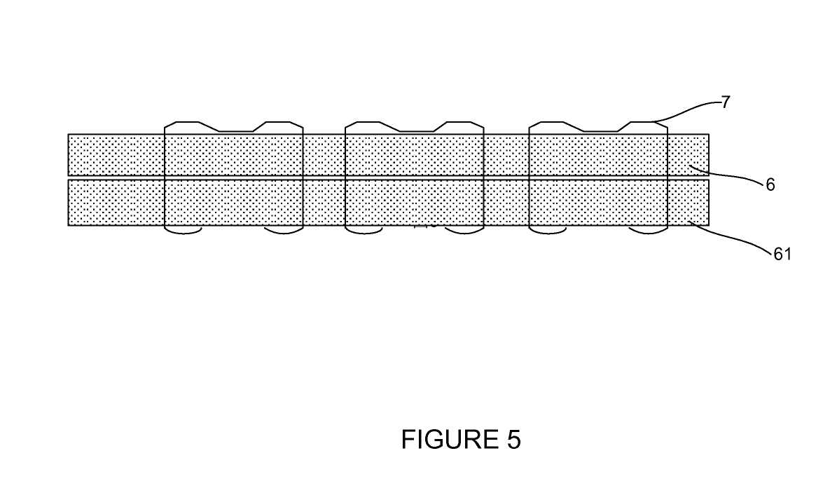

The description will be made as to the embodiments of the present invention in conjunction with the accompanying drawings in FIGS. 1-12. In accordance with the purposes of this invention, as embodied and broadly described herein, this invention, in one aspect, relates to a sandwich-type surgical structure for joining tissue.

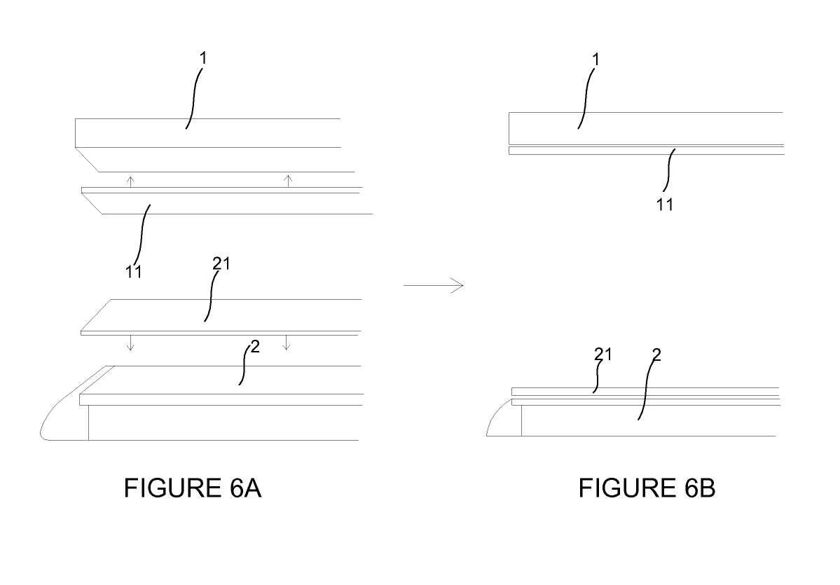

Referring initially in detail to FIGS. 6A and 6B, a sandwich-type surgical structure has first and second membrane-like structure 11 and 21 in accordance with the present disclosure intended for use with a linear stapler has a generally rectangular shape and is made of a hydrophilic biomaterial. The membrane-like structure 11 and 21 includes first surface and second surface, one of which will be in contact with the tissue being stapled, depending on whether the membrane-like structure 11 or 21 is adhered to a first member which is a nail cartridge 1 or a second member which is an anvil 2 of the nail assembly. The nail assembly operatively connected to a distal end of the body portion. As seen in FIGS. 6A and 6B, the membrane-like structure 11 or 21 has a generally rectangular cross-section.

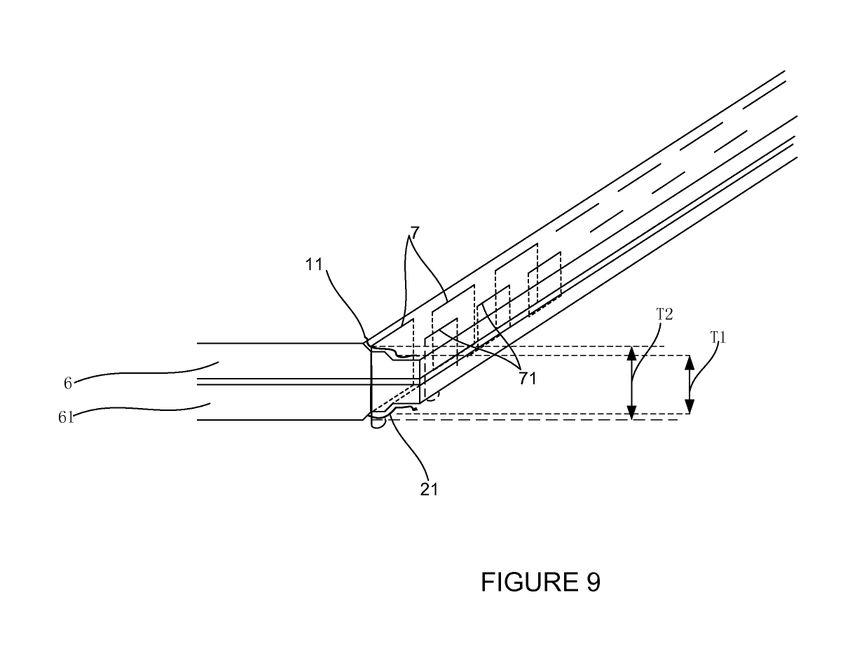

Referring again to FIGS. 6A and 6B and 6C, a sandwich-type surgical structure for joining tissue is shown according to one embodiment of the present invention. The sandwich-type surgical structure has a body portion 18 extending distally from a stationary handle portion 4. The body portion 18 defining a movable handle portion 5, an elongated central portion 3, and a nail assembly 17. The nail assembly 17 operatively connected to a distal end of the body portion 18, includes a first member as a nail cartridge assembly 1 and a second member as an anvil assembly 2. It is to be understood that the body portion 18 and the nail assembly 17 may be configured in a variety of shapes and sizes depending on a particular surgical purpose or to accommodate a particular surgical need. A release button (not shown) of a release mechanism is positioned on the proximal end of body portion 18 and is depressible to allow the nail cartridge assembly 2 to return from an approximated position, wherein the nail cartridge assembly 2 is disposed adjacent to the anvil assembly 1, to a position spaced from the anvil assembly 1 (e.g. as shown in FIG. 10).

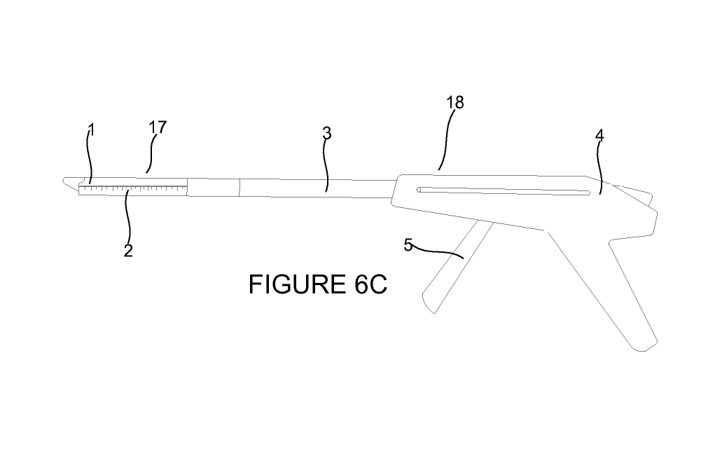

FIG. 7 shows schematically a cross-sectional view of the sandwich-type structure formed after stapling by a sandwich-type gastrointestinal anastomosis stapler according to one embodiment of the present invention. In this illustrative example, Linear surgical stapling devices are employed by surgeons to sequentially or simultaneously apply at least one or more linear rows of surgical fasteners 7, e.g., staples or two-part fasteners, to body tissue for the purpose of joining segments of body tissue together. Such devices generally include a pair of jaws or finger-like structures 72 between which body tissue to be joined is placed.

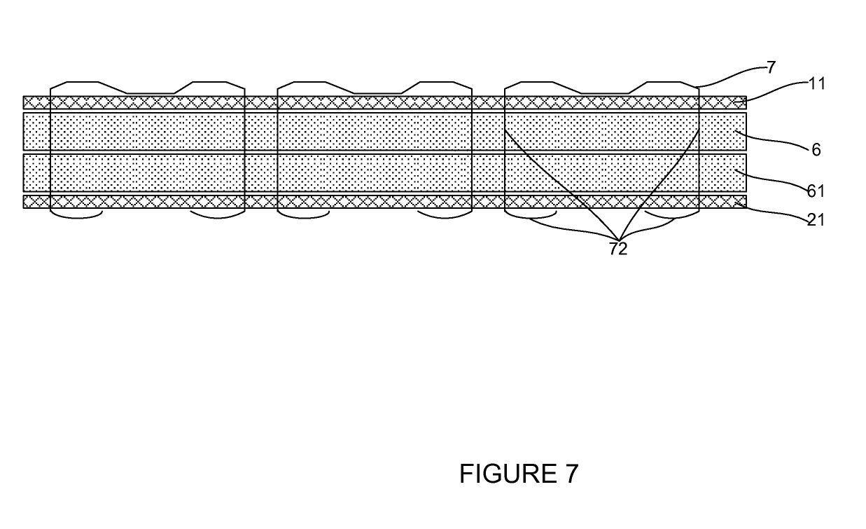

FIG. 8 shows schematically a cross-sectional view of the sandwich-type structure formed after stapling by a sandwich-type gastrointestinal anastomosis stapler with two groups of nails according to one embodiment of the present invention. In one embodiment, the legs of the staple typically pass from the cartridge jaw through a first layer of membrane-like film, then through the patient's tissue, and finally through a second layer of membrane-like film before encountering the anvil jaw. With the staples in place, the stapled tissue is clamped between the layers of membrane-like film material. Therefore, the first membrane-like structure 11, the patient's tissue 6, 61 (here is the intestine walls), and the second membrane-like structure 21 forming a sandwich structure comprising a membrane-like structure 11, the associated intestine walls 6, 61, and the membrane-like structure 21. FIG. 8 shows two groups of nails, a first group of nails 7 and a second group of nails 71, wherein the longitudinal length T2 of the nail of the second group is in the range of 5-100% length of the thickness of the sandwich-type structure before the application of the first and the second group of nails, wherein the longitudinal length T1 of the nail of the first group is in the range of 5-95% length T2 of the second group of nail, so that the force applying to the tissue will vary depending on the length of the nails. In one embodiment, before actuation of the surgical staple, the first group of nails is positioned closer to the center of the nail assembly than the second group of the nails, wherein the second group of the nail is positioned closer to the edge of the nail assembly. The shorter the length T1 of the nail, the tighter the nail will grip on the tissue; therefore, more force will apply to the tissue to keep them together therefore less leakage from stapling operation. On the other hand, the longer the length T2 of the nail, the less force squeezing the blood vessel, the more likely fluid will flow inside blood vessel to communicate nutrition to the tissue, therefore the higher likelihood the tissue can better heal with less bleeding recover from the stapling operation. In another embodiment, the length of the nail of the first group is in the range of 35-95% length of the second group of nail, so that the force applying to the tissue will vary depending on the length of the nails. In yet another embodiment, the length of the nail of the first group is in the range of 65-85% length of the second group of nail, so that the force applying to the tissue will vary depending on the length of the nails. In another embodiment, a one-part fastener, such as a regular nail, can be used, instead of two jaws or two finger-like structures are used, a nail will form just one jaw or one finger-like structure. In another embodiment, a one-part fastener, such as a regular nail, can be used, instead of two jaws or two finger-like structures are used, a nail will form just one jaw or one finger-like structure.

Again referring to FIG. 8, in one embodiment, the first member 1 is a nail cartridge assembly 1 and the second member 2 is a nail anvil assembly 2 operatively associated with the nail cartridge assembly 1. The first and second members being movably connected to one another to bring one into longitudinal juxtaposition relative to the other, wherein the first membrane-like structure immediately contacts with the tissue contacting surface of the first member of the nail assembly and wherein the second membrane-like structure immediately contacts with the tissue contacting surface of the second member of the nail assembly. When the stapling device is actuated and or “fired” firing bars move longitudinally and contact nail drive members (not shown in the Figure) in one of the jaws, surgical staples 7, 71 are pushed through the body tissue 6, 61 and into against an anvil in the opposite jaw thereby crimping the staples closed. If tissue is to be removed, a cutter blade (not shown in the Figure) can be provided to cut between the rows/lines of staples. Therefore in this embodiment, the membrane-like structure has an opening in the center portion of the membrane-like structure. Therefore, the described membrane-like structure 11, 21 can be either an annular or a strip shaped membrane, so that after the operation of the cutter, the membrane-like structure 11, 21 has an opening 50 in the center portion of the membrane-like structure 11, 21.

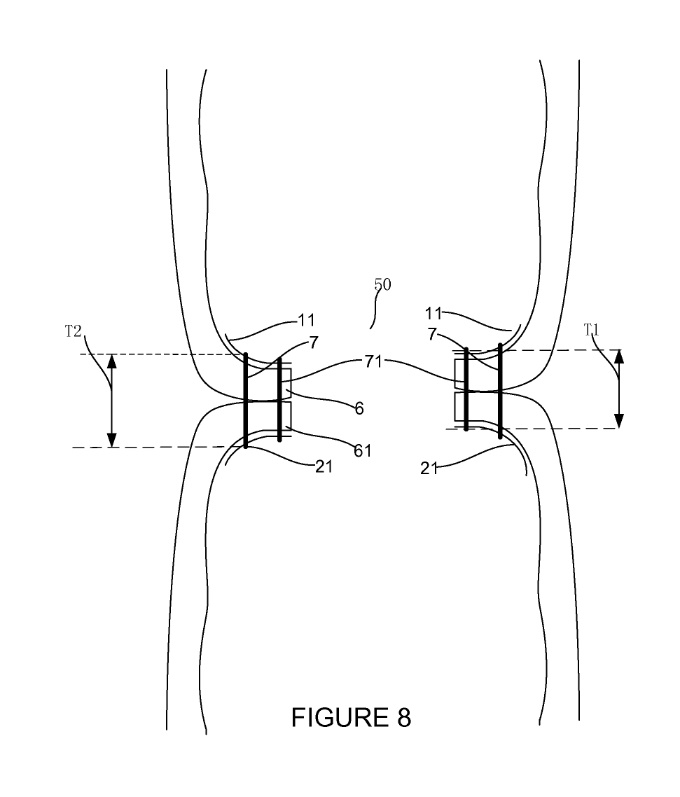

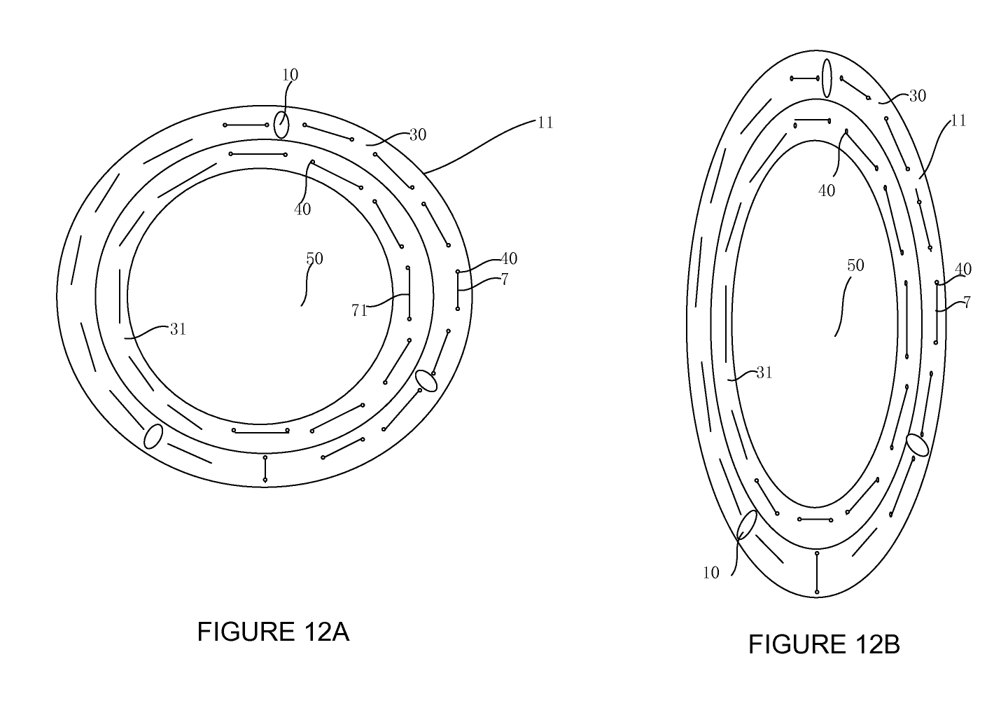

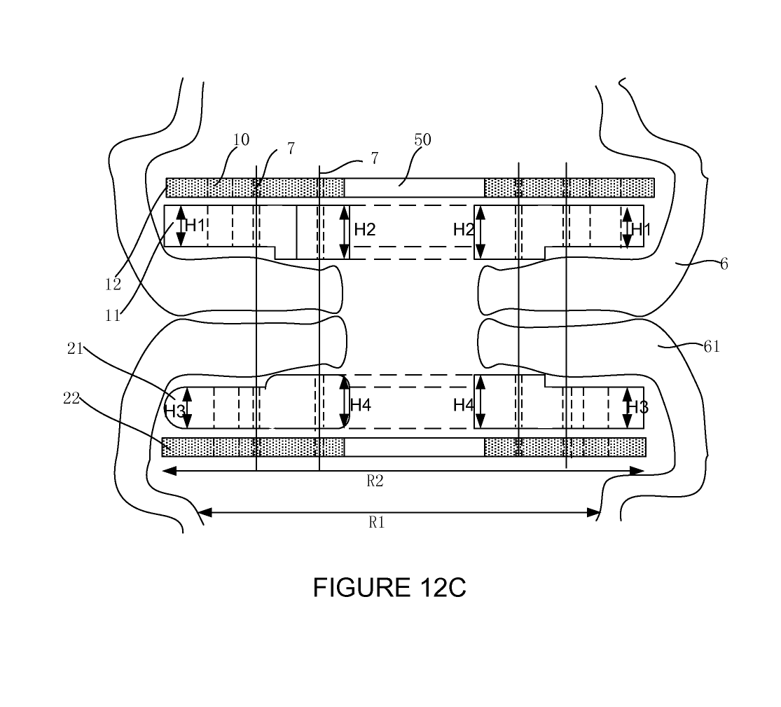

FIG. 9 shows schematically a perspective view of the sandwich-type structure formed after stapling by a sandwich-type gastrointestinal anastomosis stapler with two groups of nails according to one embodiment of the present invention. In one embodiment, the first and second groups of nails are linear arrays, wherein the linear arrays parallel to each other; wherein the first and second linear arrays are staggered to each other. The staggerly arrangement of the two groups of nail can also be shown in FIGS. 12A and 12B. Due to the stagger positioning, the non sealed portion of the tissue between neighboring staple of the first group of staples will be sealed by the second groups of staples. Due to alternating and overlapping positions by stagger, wherein the leading edge of the first group staples is between the leading edge and second edge of number N of the second group of the staples, wherein the second edge of the first group staples is also between the number N+1 staple the leading edge and second edge of the second group of the staples, wherein the N and N+1 staple are immediately neighboring ones within the same group of staples. It is desirable that the opening between the neighboring nails of the first and second group of the nails is less than the length between the two jaws of one nail. The length between the two jaws of one nail has typical range of value from 25 to 75 mm in the present invention; the opening between the neighboring nails of the first and second group of the nails is typically between 10 to 20 mm By staggerly positioned the staples, each opening between staples will be fully overlapped by another group of nails with the clamping force provided by the two jaws and the connecting bar between the two jaws. The stagger will ensure a proper sealing action of the nails will be continuous without leakage throughout the joining area of the tissue that is joined.

FIG. 10 shows schematically a cross sectional view of the sandwich-type structure formed after stapling by a sandwich-type gastrointestinal anastomosis stapler with two groups of nails according to one embodiment of the present invention. In one embodiment, the first and second groups of nails are circular arrays distributing around the annular staple, wherein there are two groups of concentrically circular arrays; wherein the first and second linear arrays are interposing to each other. The interposing of the two groups of nail can also be shown in FIGS. 12A and 12B. Due to the interposing, the openings 30, 31 of one group of nail will be close by the sealing action of the other groups of nail. It is desirable that the opening 30, 31 between the neighboring nails of the first and second group of the nails is less than the length between the two jaws of one nail. The length between the two jaws of one nail has typical range of value from 25 to 75 mm in the present invention; the opening 30, 31 between the neighboring nails of the first and second group of the nails is typically between 10 to 20 mm. By interposing, these openings 30, 31 will fully overlap by another group of nails with the clamping force provided by the two jaws and the connecting bar between the two jaws. The interposing will ensure a proper sealing action of the nails will be continuous without leakage throughout the joining area of the tissue that is joined.

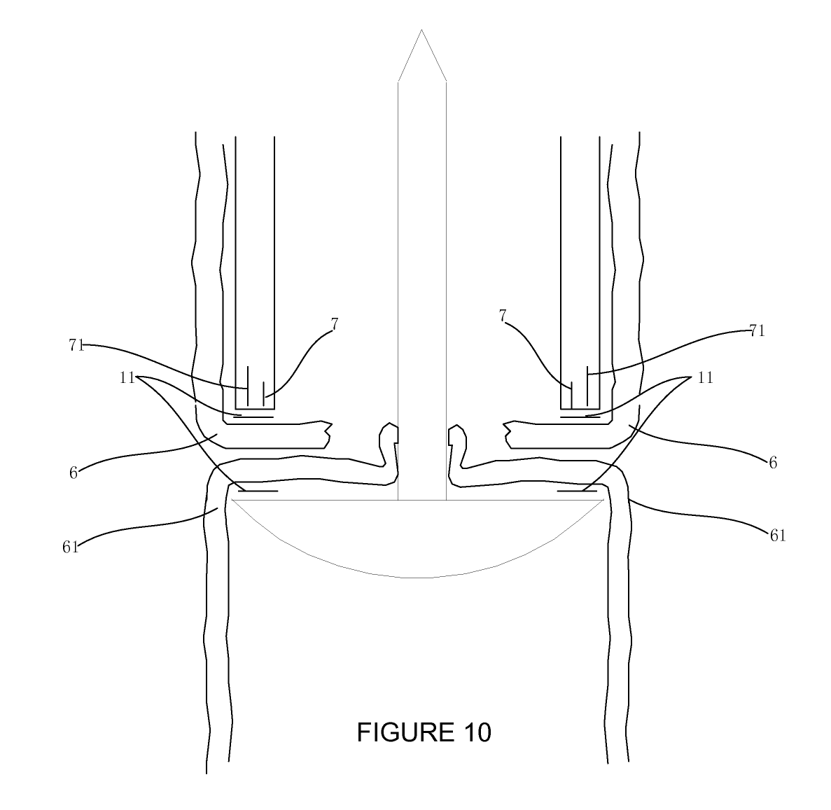

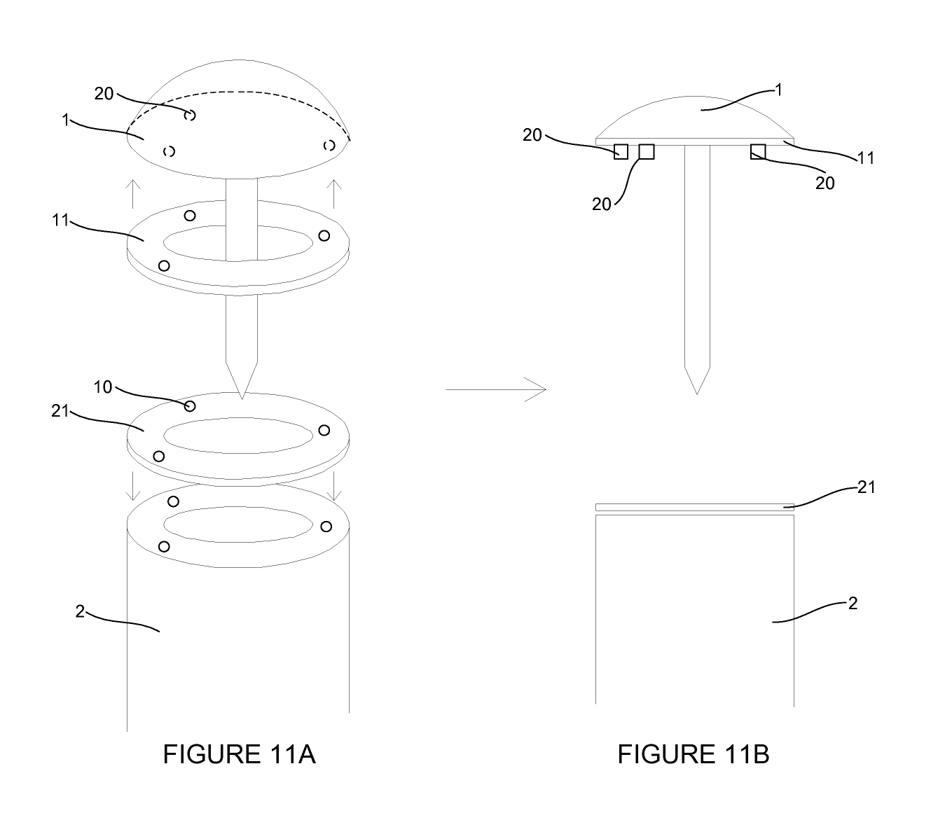

FIG. 11A show schematically perspective views of (for annular stapler-type sandwich gastrointestinal anastomosis cutting stapler) the nail anvil; nail assembly and membrane-like's structure according to one embodiments of the present invention; FIG. 11B show schematically cross-sectional views of (for annular stapler-type sandwich gastrointestinal anastomosis cutting stapler) the nail anvil; nail assembly and membrane-like's structure according to one embodiments of the present invention. FIGS. 12A and 12B shows schematically a perspective view of the sandwich-type structure formed after stapling by a sandwich-type gastrointestinal anastomosis stapler with two groups of nails according to one embodiment of the present invention. In one embodiment, the membrane-like structure has multiple openings 40 aligned with the first and second groups of nails 7, 71. When the staples are applied in surgical operation utilizing membrane-like film, the legs of the staple typically pass from the cartridge jaw through the nail opening 40 aligned with the individual nail of the first layer of membrane-like film, then through the patient's tissue before encountering the anvil jaw. In another embodiment, the membrane-like structure may have positioning hole 10 to align the nail assembly with the membrane-like structure by align the positioning hole 10 with a positioning post 20 (as shown in FIGS. 11A and 11B) in the anvil assembly.

FIGS. 12A and 12B shows schematically a perspective view of the sandwich-type structure formed after stapling by a sandwich-type gastrointestinal anastomosis stapler with two groups of nails according to one embodiment of the present invention. The membrane-like film above can be used in conjunction with linear surgical staplers or with annular surgical staplers. The membrane-like film can be any shape, and will normally be configured to correspond to and cover at least a portion of a staple line applied by a surgical stapler. Suitable shapes include rectangular strips (e.g., for linear staplers) and annular rings (e.g., for annular staplers). The cross-sectional shape of the support structure has any cross-sectional profile, such as, for example, generally rectangular, circular (e.g. FIG. 12A), ovoid (e.g. FIG. 12B), triangular, arcuate, etc.

The thickness of the membrane-like film is controlled by the volume of the monomer composition polymerized in the mold. Suitable thickness for the membrane-like film is in the range of about 0.1 to about 5 mm in one embodiment. In yet another embodiment, the annular shaped membrane-like film has a width of 1.5˜3.0 mm, and a thickness of 0.1˜1.0 mm. In another embodiment, the strip shaped membrane has a width of 1.5˜5 mm, and a thickness of 0.1˜1.0 mm.

In one embodiment, it is contemplated that the membrane-like structure has both flexibility and rigidity. The membrane-like structure has flexibility to adapted to the surface of the biological tissue, at the same time, it will have rigidity to compress the tissue, to hold the nails in place.

In one embodiment, the membrane-like structure uses a degradable polymer as the material. In another embodiment, the membrane-like structure uses a non-degradable polymer as the material. In yet another embodiment, the membrane-like structure uses one of the silicone, poly(lactic-co-glycolic acid, poly lactic acid, polyoxyethylene.

FIG. 12C shows schematically a cross-sectional view of the sandwich-type structure formed after stapling by a sandwich-type gastrointestinal anastomosis stapler with two groups of nails according to one embodiment of the present invention. In one embodiment, the membrane-like structure has at least two thicknesses defining two areas, shown as H1 and H2 of the membrane-like structure 11, and H3 and H4 of the membrane-like structure 21. In one embodiment, the longitudinal length H1 of the nail of the membrane-like structure is in the range of 5-95% length H2 of the membrane-like structure, so that the force applying to the tissue will vary depending on the thickness of the membrane-like structure. In another embodiment, the longitudinal length H1 of the nail of the membrane-like structure is in the range of 35-95% length H2 of the membrane-like structure, so that the force applying to the tissue will vary depending on the thickness of the membrane-like structure. In yet another embodiment, the length of the nail of the first group is in the range of 65-85% length of the second group of nail, so that the force applying to the tissue will vary depending on the length of the nails. The thicker area will be closer to the central area of a nail assembly. The thicker the membrane-like structure, the larger the squeezing force will apply on the tissue from the same length of a nail. In one embodiment, the thicker area will be closer to the opening from the operation of the cutter, so that the force applying to the tissue will vary depending on the thickness of the membrane-like structure.

In yet another embodiment, a sandwich-type surgical structure for joining tissue, in additional to the variation of thickness of the membrane-like structure, the length of the nail of the first group is in the range of 5-95% length of the second group of nail, so that the force applying to the tissue will vary depending on the length of the nail and the thickness of the membrane-like structure.

In another embodiment, it is contemplated that a uniform thickness membrane-like structure can be used, however, at the place where the first group of nails are used, the structure may have recess holes, so that the effective thickness of the membrane-like structure is thinner dependent on the effective thickness due to the thickness reduction by the recess holes, which is similar to have a different thickness of membrane-like structure.

Again refereeing to FIG. 12C, in one embodiment, additional layers 10, 22, can be in additional to the membrane-like structure 11, 21. Such layers can be non biodegradable, so that it can remain integration with the nails at the end. Anastomotic nail may fall off with the double-membrane-like, so as to remove external surgical object nail, therefore, reducing the possibility of anastomotic stricture and inflammation occurrence. The additional layer 10, 22 will be bio-nondegradable, has certain hardness and toughness against various chemical reactions. The additional layer can prevent the unlikely event that the double-membrane-like was not strong enough to retain integration with the groups of nail to happen, as they will remain strong even after the double-membrane-like weaken due to its interactions with the biological environment.

Although the present invention does a few structures improvement, it not only solves the long-term common problems of gastrointestinal surgical anastomosis tearing, anastomotic leakage, anastomotic bleeding and anastomotic section bleeding, but also it has advantages of low cost and shorter operation time, therefore it has unexpected technical advantages.

In another embodiment, a method for joining tissue is disclosed. The method comprising the steps of: providing a sandwich-type surgical structure for joining tissue, comprising: a movable handle portion; a body portion extending distally from the handle portion; a nail assembly operatively connected to a distal end of the body portion, the nail assembly comprising: a first member and a second member; at least first and second arrays of nails, wherein the length of the nail of the first array is in the range of 5-95% length of the second array of nail, so that the force applying to the tissue will vary depending on the length of the nails; and a first membrane-like structure wherein the first membrane-like structure immediately contacts with the tissue contacting surface of the first member of the nail assembly, (placed in longitudinal juxtaposition to at least one of a tissue contacting surface of the first member); providing a first and a second tissue to be jointed; implanting first and second arrays of nails into the membrane-like structure and the tissue to be jointed using the nail cartridge assembly, thereby affixing the membrane-like structure to the first and the second tissue to be jointed; positioning the first membrane-like structure adjacent a tissue contacting surface of a first member of the nail cartridge assembly;

In another embodiment, the method for joining tissue including moving the first member relative to the second member, thereby bringing a first membrane-like structure adjacent to the tissue to be jointed;

In one embodiment, the step of positioning the first membrane-like structure adjacent a tissue contacting surface of a first member of the nail cartridge assembly further comprising after the step of positioning the second membrane-like structure adjacent a tissue contacting surface of the second member of the nail cartridge assembly; positioning the tissue to be jointed between a second member of the nail cartridge assembly and the second membrane-like structure;

In another embodiment, the step of providing the tissue to be jointed includes at least one of selecting and preparing the tissue to be jointed for use in a surgical procedure, further comprising the step of: aligning the tissue to be jointed to facilitate surgical fastener placement within a center portion of the tissue.

In yet another embodiment, the method for joining tissue including the step of removing the first group and second group of staples entirely from the tissue through the integration force between the staples and the first and the second membrane-like structures, wherein the first and the second membrane-like structures will have at least a portion of rigid component that will last longer before the staples disengaged from the tissue; whereby the staples can be removed from the tissue entirely through the residual attachment force between the membrane-like structures of the staples with the first and the second group of membrane-like structures.

In brief, the present invention, among other things, recites a surgical structure having a plurality of groups of nails spatially arranged next to each other such that at least one group has a length shorter than the other group. Comparing with a conventional surgical stapler, the invented photovoltaic module has advantages in preventing gastrointestinal anastomotic tear, anastomotic leakage and anastomotic bleeding, therefore a better tissue healing.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

What is claimed is:

1. A sandwich-type surgical structure for joining tissue, comprising:
a movable handle portion;
a body portion extending distally from the handle portion;
a nail assembly operatively connected to a distal end of the body portion,
the nail assembly comprising:
a first member;
a second member;
a first group of nails;
a second groups of nails, wherein the length of the nail of the first group is in the range of 5-95% length of the second group of nail, so that the force applying to the tissue will vary depending on the length of the nails; and
a first membrane-like structure wherein the first membrane-like structure immediately contacts with the tissue contacting surface of the first member of the nail assembly.

2. The sandwich-type surgical structure of claim 1, wherein the sandwich-type surgical structure further comprising:
a second membrane-like structure wherein the second membrane-like structure immediately contacts with the tissue contacting surface of the second member of the nail assembly.

3. The sandwich-type surgical structure of claim 2, wherein the first membrane-like structure, the associated intestine walls, and the second membrane-like structure forming a sandwich structure comprising the first membrane-like structure, associated intestine walls, and the second membrane-like structure.

4. The sandwich-type surgical structure of claim 1, wherein the described membrane-like structure is an annular or a strip shaped membrane; wherein the membrane-like structure has an opening in the center portion of the membrane-like structure, wherein the membrane-like structure has multiple openings aligned with the first and second groups of nails.

5. The sandwich-type surgical structure of claim 1, wherein the annular shaped membrane has a width of 1.5˜3.0 mm, and a thickness of 0.1˜1.0 mm.

6. The sandwich-type surgical structure of claim 1, wherein the strip shaped membrane has a width of 1.5˜5 mm, and a thickness of 0.1˜1.0 mm.

7. The sandwich-type surgical structure of claim 1, wherein the membrane-like structure has both flexibility and rigidity.

8. The sandwich-type surgical structure of claim 7, wherein the membrane-like structure with flexibility and rigidity is a degradable polymer.

9. The sandwich-type surgical structure of claim 7, wherein the membrane-like structure with flexibility and rigidity is a non-degradable polymer.

10. The sandwich-type surgical structure of claim 1, wherein the membrane-like structure is one of the silicone, polymer milk alcohols, poly lactic acid, polyoxyethylene, polyester material.

11. The sandwich-type surgical structure of claim 1, wherein the sandwich-type surgical structure further comprising:
a cutter for cutting excess tissues from an associated digestive tract to open up a central conduit to complete the reconstruction of the associated digestive tract;
wherein the first and second group of nails are linear arrays, wherein the linear arrays parallel to each other; wherein the first and second linear arrays are interposing, wherein the second member is operatively associated with the first member, the first and second members being movably connected to one another to bring one into longitudinal juxtaposition relative to the other.

12. The sandwich-type surgical structure of claim 1, wherein the first member is a nail cartridge assembly and the second member is a nail anvil assembly operatively associated with the nail cartridge assembly.

13. The sandwich-type surgical structure of claim 1, wherein the length of the nail of the first group is in the range of 35-95% length of the second group of nail, so that the force applying to the tissue will vary depending on the length of the nails.

14. A sandwich-type surgical structure for joining tissue, comprising:
a movable handle portion;
a body portion extending distally from the handle portion;
a nail assembly operatively connected to a distal end of the body portion,
the nail assembly comprising:
a first member;
a second member;
a first group of nails;
a second groups of nails, wherein the length of the nail of the first group is in the range of 5-95% length of the second group of nail, so that the force applying to the tissue will vary depending on the length of the nails.

15. The sandwich-type surgical structure of claim 15, wherein the sandwich-type surgical structure further comprising:
a cutter for cutting excess tissues from an associated digestive tract to open up a central conduit to complete the reconstruction of the associated digestive tract;
wherein the first and second group of nails are linear arrays, wherein the linear arrays parallel to each other, wherein the first and second linear arrays are interposing; wherein the second member is operatively associated with the first member, the first and second members being movably connected to one another to bring one into longitudinal juxtaposition relative to the other.

16. The sandwich-type surgical structure of claim 15, wherein the first member is a nail cartridge assembly and the second member is a nail anvil assembly operatively associated with the nail cartridge assembly.

17. The sandwich-type surgical structure of claim 15, wherein the length of the nail of the first group is in the range of 35-95% length of the second group of nail, so that the force applying to the tissue will vary depending on the length of the nails.

18. The sandwich-type surgical structure of claim 15, wherein the length of the nail of the first group is in the range of 65-85% length of the second group of nail, so that the force applying to the tissue will vary depending on the length of the nails.

19. A sandwich-type surgical structure for joining tissue, comprising:
a movable handle portion;
a body portion extending distally from the handle portion;
a nail assembly operatively connected to a distal end of the body portion,
the nail assembly comprising:
a first member;
a second member;
a first group of nails;
a second groups of nails; and
a first membrane-like structure wherein the first membrane-like structure immediately contacts with the tissue contacting surface of the first member of the nail assembly, wherein the first membrane-like structure has at least two thicknesses.

20. A sandwich-type surgical structure for joining tissue of claim 19, wherein the length of the nail of the first group is in the range of 5-95% length of the second group of nail, so that the force applying to the tissue will vary depending on the length of the nail and the width of the membrane-like structure.
